(19) United States
(12) Patent Application Publication
(10) Pub. No.: US 2008/0069843 A1
(43) Pub. Date: Mar. 20, 2008

(54) Salmonella vaccine

(75) Inventors: Petrus J.M. Nuijten, Boxmeer (NL); Maarten H. Witvliet, Oostrum (NL)

Correspondence Address: TRASK BRITT, P.O. BOX 2550, SALT LAKE CITY, UT, 84110, US

(73) Assignee: Intervet International B.V., Boxmeer (NL)

(21) Appl. No.: 11/980,864

(22) Filed: Oct. 30, 2007

Related U.S. Application Data
(62) Division of application No. 09/749,025, filed on Dec. 27, 2000.

(30) Foreign Application Priority Data
Dec. 28, 1999 (EP) 99204564.1

(51) Int. Cl. A61K 39/112

(52) U.S. Cl. 424/258.1

(57) ABSTRACT

The present invention relates to Salmonella bacteria. The invention also relates to methods of using Salmonella bacteria, including in vaccines based thereon that are useful for the prevention of microbial pathogenesis. Further, the invention relates to the use of such bacteria or the manufacture of such vaccines. The invention also relates to methods for the preparation of such vaccines.

### CROSS-REFERENCE TO RELATED APPLICATIONS

This application is a divisional of U.S. patent application Ser. No. 09/749,025, filed Dec. 27, 2000, pending, which application claims priority to, and is a continuation of, European Patent Application No. 99204564.1 filed on Dec. 28, 1999, the contents of each of which are hereby incorporated herein by this reference.

### TECHNICAL FIELD

The present invention relates to Salmonella bacteria for use in a vaccine, to vaccines based upon these bacteria, to the use of such bacteria for the manufacture of a vaccine and to methods for the preparation of such vaccines.

### BACKGROUND

Bacteria of the genus Salmonella are notorious for their pathogenicity in both man and animals. In the USA alone, on a yearly basis the number of humans suffering from Salmonella infections exceeds the two million cases. In most cases, the infection is caused by contaminated food. Well-known sources of infection are eggs (from both ducks and chickens), products containing eggs and insufficiently heated poultry and pig meat. Especially in infants, young children, elderly people and immune compromised patients, the ability to cope with such infections is low. Especially in these groups, the yearly death rate due to Salmonella infections is high. During the last few decades more efficient large-scale animal husbandry has led to an enormous increase in animal density. As a result, an increase has been seen in the number of animal infections and subsequent human infections caused by infected food. It is clear that animals are the main source of Salmonella infection. This source is very difficult to control. First, Salmonella infections in most cases cause no serious illness in healthy full-grown animals; these animals can carry the bacterium for a prolonged period. During that time, they are shedding the bacterium in their dung. This makes it practically impossible to avoid infection in the more vulnerable young animals. Secondly, many Salmonella species colonize several different host animal species. Some of the Salmonella species cause primary infections in specific hosts, whereas other Salmonella species are not restrictive at all. As a primary infectans, S. typhi and paratyphi are frequently associated with infection in man. S. typhi causes diarrhea, and as a result, dehydration. This infection is very frequently found in tropical areas. S. dublin is connected with cattle, specifically young animals, where it causes lethal infections in over 50% of the cases. S. abortus-equi causes abortion in horses. S. abortus-ovi causes abortion in sheep. S. choleraesuis is the cause of lethal diarrhea in young pigs. S. typhimurium and S. enteritidis cause salmonellosis in poultry, pigs, cattle and rodents. S. arizonae also causes disease in turkeys. Also, non-food animals such as reptiles suffer from Salmonella infections, such as S. arizonae.

There clearly is a need for efficient Salmonella vaccines. Vaccines are needed to protect humans from Salmonella infections transmitted from man to man. Also, vaccines are needed to protect humans from food-borne Salmonella infections. And finally, vaccines are needed to protect animals against Salmonella infections.

Currently, several vaccines against various Salmonella species are commercially available. These vaccines, although sometimes efficacious from a vaccine point of view, share a serious disadvantage. They generally induce an antibody population that equals that of an infection with wild-type bacteria because they possess the same antigenic load as the wild-type bacterium. Therefore, an analysis of the antibodies in the serum of a Salmonella-positive animal does not reveal why the animal is positive. This can be due to vaccination, but it can equally be caused by infection with a virulent field strain. Therefore, if an animal is Salmonella-positive, it is considered to be infected. Therefore, it would be desirable to have a so-called marker vaccine. A marker vaccine is a vaccine that can be discriminated from wild-type infection, for example, on the basis of a characteristic antibody panel, different from the antibody panel induced by wild-type infection. A different antibody panel is induced, for example, when an immunogenic protein present in a wild-type bacterium is not present in a mutant that is used for vaccination. The host will then not make antibodies against that protein after vaccination.

A marker antigen has at least the following characteristics:

it can be deleted without severely impairing the viability of the bacterium;

it induces antibodies when present;

it does not, when present, contribute to the immunogenicity of the bacterium;

its absence preferably contributes to attenuation.

When searching for suitable marker antigens, all known Salmonella antigens are to be considered. A known antigen found with all wild-type Salmonella species with the exception of some S. pullorum and gallinarum subspecies is the flagellum. Examples of Salmonella species carrying flagella when in their wild-type form are S. typhimurium, enteritidis, choleraesuis, dublin, typhi, abortus-ovi, abortus-equi, paratyphi A and B, derby, hadar, heidelberg, agona and arizonae. Flagella are long structures protruding from the cell surface that play an important role in motility and invasion of certain host cells. Flagella consist of long polymers of the protein called flagellin. It is known that these flagella induce high levels of antibodies. It is known that the absence of flagella does not significantly impair the viability of the bacterium outside the host: flagella-less mutants of practically all Salmonella species are known and can be grown in vitro. Nevertheless, flagellar proteins of Salmonella have never been contemplated as suitable markers, since they do not, or only partially, fulfill three of the four marker-requirements:
although not essential for survival outside the host, they do play a significant role in bacterial survival and persistence in host macrophages, involved in inducing immunity (U. Methner and P. A. Barrow, Berl. Münch. Tierärtzl. Wschr. 110:391-396 (1997); Weinstein et al., Infect. Immun. 46: 819-825 (1984));
they strongly contribute to the immunogenicity of the bacterium, a reason why they are used as carrier proteins for short heterologous amino acid sequences (T. M. Joys, SAAS Bulletin: Biochem. & Biotech. 4:56-59 (1991); S. M. C. Newton et al., Science 244:70-72 (1989));
their absence does not contribute to attenuation (H. A. Lockman and R. Curtiss, Infect. & Immun. 58:137-143 (1990); U. Methner and P. A. Barrow, Berl. Münch. Tierärtzl. Wschr. 110:391-396 (1997));
flagellated Salmonella bacteria inhibit the binding of other flagellated Salmonellae. Flagella-less Salmonella bacteria however show a reduced inhibition potential towards other flagellated Salmonellae, and therefore even increase the overall level of Salmonella infection (U. Methner and P. A. Barrow, Berl. Münch. Tierärtzl. Wschr. 110:391-396 (1997); Weinstein et al., Infect. Immun. 46:819-825 (1984)).

For these combined reasons, the flagellum was never considered to be a suitable deletable marker antigen for Salmonella vaccines.

### DISCLOSURE OF THE INVENTION

Surprisingly, it was found now that contrary to the general assumption, the absence of flagella did not affect the vaccinating potential of Salmonella. This is especially surprising in view of the fact that in wild-type Salmonella infected animals, large amounts of antibodies against the flagella are found, once more proving their antigenicity.

One aspect of the present invention provides, for the first time, bacteria of the genus Salmonella that in their wild-type form carry flagella but are no longer capable of inducing antibodies against at least one antigenic determinant of flagellin or flagella, for use in a vaccine for the protection of humans and animals against Salmonellosis.

Flagellins are proteins of about 500 amino acids that comprise several antigenic determinants, i.e., there are several regions on the flagellins against which antibodies are raised in the host animal. These antigenic determinants have been identified and localized by T. M. Joys (T. M. Joys, SAAS Bulletin: Biochem. & Biotech. 4:56-59 (1991)). For the purpose of the present invention, bacteria that are no longer capable of inducing antibodies against at least one antigenic determinant of flagellin or flagella are considered to be bacteria that do not comprise flagellin or flagella that still possesses all the antigenic determinants. It is not necessary to remove all antigenic determinants: it suffices to delete one of the antigenic determinants. Screening for antibodies against this antigenic determinant in the serum of seropositive animals would then allow discrimination between marker-vaccinated and wild-type infected animals. Such antibodies would not be found in vaccinated animals, but would be present in naturally infected animals. Screening can easily be done with simple diagnostic tools on a routinely basis as will be described below.

Much is currently known about the synthesis of flagellin and the subsequent maturation into flagella (for example, in Escherichia coli and Salmonella typhimurium, Chapter 10: Flagella and motility, eds. Frederick C. Neidhardt et al. 2nd. ed. ISBN 1-55581-084-5 (1996); and in R. M. Macnab, Genetics and biogenesis of bacterial flagella (Annual Review of Genetics 26:131-158 (1992)). The process of flagellar biogenesis requires the concerted action of a large number of genes, not only the gene encoding the flagellin but also a large number of genes involved in the synthesis of the flagellum and the flagellar motor. In principle, there are two approaches for making bacteria according to the present invention. First, mutations can be made in the gene encoding the flagellin protein in order to mutate one or more antigenic determinants. Secondly, one or more genes involved in the biogenesis of the flagellum can be mutated. This will prevent the biosynthesis of flagellin or even the whole flagellum, and in many cases even the transport of the flagellin through the bacterial membrane. In the case that no flagella are produced, antigenic determinants determined by the specific folding of the flagellin in flagella will be absent. If the flagellin itself is not transported through the membrane and thus remains inside the bacterium, there will be no induction of antibodies against flagellin. All kinds of genes or gene clusters known in the art to be involved in assembly and export of such flagella as the Morphological Assembly Pathway and a Flagellum-specific Export Pathway, can be targets for mutation as they all lead either to bacteria lacking flagellin or at least flagella. For the sake of clarity, all pathways involved in the whole process of synthesis of the final flagella are further referred to as flagellar biogenesis pathways.

Therefore, in a preferred embodiment, the bacterium is not capable of inducing antibodies against at least one antigenic determinant of flagellin or flagella due to a mutation in a gene of the flagellar biogenesis pathway.

From a practical point of view, it may however be desirable to delete (part of) the whole flagellin gene from the bacteria to be used in the vaccine, simply by deletion of the gene encoding the flagellar protein. The genes encoding the flagellar proteins of the various Salmonella species are known. They are all very closely related, and therefore highly homologous. Flagellin genes have, i.e., been described for Salmonella enterica (J. Li et al., Proc. Natl. Acad. Sci. 91:2552-2556 (1994)), Salmonella enteritidis (R. K. Selander et al., J. Bacteriol. 174:3587-3592 (1992)), Salmonella dublin (B. J. Masten and T. M. Joys, J. Bacteriol. 175:5359-5365 (1993)), Salmonella typhimurium (N. de Vries et al., Appl. Environ. Microbiol. 64:5033-5038 (1998)), Salmonella abortus-equi (T. Hanafusa et al., Mol. Gen. Genet. 236:260-266 (1993)). Flagellin genes of novel Salmonella species can easily be found on the basis of their homology with all existing and known Salmonella flagellin genes: standard hybridization techniques suffice for locating the flagellin gene.

There are two flagellin genes in wild-type flagellum-bearing Salmonella bacteria. Only one of these genes is switched on, i.e., produces flagellin at any given time. Due to a mechanism called flagellar phase variation, at a time scale on the order of 10³ to 10⁵ generations the genes change roles so that the non-expressed one becomes expressed and vice versa (for example, described in Escherichia coli and Salmonella typhimurium, Chapter 10: Flagella and motility, eds. Frederick C. Neidhardt et al. 2nd. ed. ISBN 1-55581-084-5 (1996)). In order to avoid strains according to the invention that start expressing flagellin after a certain number of generations, both flagellin genes must be made non-functional. Alternatively, if the phase switching mechanism is made non-functional it suffices to knock out the flagellin gene that is expressed at that time.

A non-functional gene is a gene that no longer encodes a flagellin. This can be a gene from which a part of the coding sequence has been removed that encodes an antigenic determinant.

One possible way of making the flagellin gene or any of the other known genes involved in flagellum-biosynthesis non-functional is by means of classical methods such as the treatment of wild-type bacteria flagella-producing bacteria with mutagenic agents such as base analogs, treatment with ultraviolet light, or temperature treatment (P. Anderson, 1995, Mutagenesis, p 31-58, in Methods in Cell Biology 48, H. F. Epstein and D. C. Shakes (Eds)). Other methods for making flagella-less mutants of various bacteria of which the wild-type has flagella, have been described, i.e., by S. L. Liu et al. (Infect. Immun. 1988 August; 56(8):1967-73), R. Haas et al. (Mol. Microbiol. 1993 May; 8(4):753-60), and J. Graf et al. (J. Bacteriol. 1994 November; 176(22):6986-91).

Selection for flagella-less bacteria is very easily done by light microscopic analysis for the absence or presence of flagella. Selection for bacteria lacking at least one antigenic determinant of flagellin or flagella can be done by means of binding assays with monoclonal antibodies against flagellin or flagella. Such anti-flagellar or anti-flagellin monoclonal antibodies can be made according to standard techniques, i.e., by immunizing (with flagella) inbred mice by techniques known in the art (Kohler and Milstein, Nature, 256:495-497, 1975). Monoclonals thus obtained can be used in binding assays with the mutated bacteria, and those bacteria not binding to a specific monoclonal are the bacteria lacking at least one antigenic determinant of flagellin or flagella.

The nature of the mutation caused by classical mutation techniques is unknown. This may be a point mutation which may, although this is unlikely to happen, eventually revert to wild-type. Therefore transposon mutagenesis is a good alternative. Mutation by transposon mutagenesis, is also a mutagenesis-technique well-known in the art. This is a mutation accomplished at a localized site in the chromosome.

A possibility to introduce a mutation at a predetermined site, deliberately rather than randomly, is offered by recombinant DNA-technology. Such a mutation may be an insertion, a deletion, a replacement of one nucleotide by another one or a combination thereof, with the only proviso that the mutated gene no longer encodes functional flagellin. Such a mutation can, for example, be made by deletion of a number of base pairs. Even very small deletions such as stretches of ten base pairs can already render flagellin non-functional. Even the deletion of one single base pair may lead to a non-functional flagellin, since as a result of such a mutation, the other base pairs are no longer in the correct reading frame. Each deletion or insertion of a number of base pairs indivisible by three causes such a frame shift. More preferably, a longer stretch is removed, for example, 100 base pairs. Even more preferably the whole flagellin gene is deleted. It can easily be seen, that especially mutations introducing a stop-codon in the open reading frame, or mutations causing a frame-shift in the open reading frame are very suitable to obtain a strain which no longer encodes flagellin. Site-directed mutagenesis is the method of choice for making a flagellin gene that lacks one or more specific antigenic determinants. Based upon the map of antigenic determinants made by Joys (T. M. Joys, SAAS Bulletin: Biochem. & Biotech. 4:56-59 (1991)), mutant flagellin genes can be made from which one or more specific antigenic determinants have been removed. All recombinant DNA techniques for the construction of flagellin-negative mutants are well-known standard techniques. They relate to cloning of the flagellin-gene, modification of the gene sequence by site-directed mutagenesis, restriction enzyme digestion followed by re-ligation or PCR-approaches and to subsequent replacement of the wild-type flagellin gene with the mutant gene (allelic exchange or allelic replacement). Standard recombinant DNA techniques such as cloning the flagellin gene in a plasmid, digestion of the gene with a restriction enzyme, followed by endonuclease treatment, re-ligation and homologous recombination in the host strain, are all known in the art and described i.e., in Maniatis/Sambrook (J. Sambrook et al., Molecular cloning: a laboratory manual, ISBN 0-87969-309-6). Site-directed mutations can, for example, be made by means of in vitro site-directed mutagenesis using the Transformer® kit sold by Clontech. PCR-techniques are extensively described in (C. Dieffenbach & G. Dveksler, PCR primers, a laboratory manual, CSHL Press, 1995, ISBN 0-87969-447-3).

Therefore, in a preferred form, an embodiment of the invention relates to a bacterium that is not capable of expressing a flagellin due to a mutation in the flagellin gene.

Most preferably, the bacteria are selected from the group consisting of S. typhimurium, enteritidis, choleraesuis, dublin, typhi, abortus-ovi, abortus-equi, paratyphi A and B, derby, hadar, Heidelberg, agona and arizonae.

It is another object of the present invention to provide for the first time suitable marker vaccines for the protection of humans and animals against Salmonellosis. Vaccines according to the invention have as a characteristic feature that they comprise bacteria as defined above or antigenic material thereof, and a pharmaceutically acceptable carrier. The main advantage of vaccines according to the present invention is, that humans and animals vaccinated therewith can be discriminated from both non-vaccinated humans and animals and wild-type Salmonella infected humans and animals on the basis of their antibody panel.

Vaccines according to the invention can be in a live attenuated or an inactivated form. In both cases the absence of at least one antigenic determinant of flagella or flagellin will result in an antibody panel after vaccination that can easily be discriminated from that induced after wild-type infection.

Inactivated vaccines have the advantage over live vaccines that they are inherently safe. Therefore, one preferred form of the invention relates to vaccines in which the bacteria are in an inactivated form.

Vaccines according to the invention that are based upon live Salmonella bacteria have an advantage over vaccines comprising inactivated bacteria because they better mimic the natural infection and therefore trigger the immune system in a better way. They also have an additional important advantage as explained hereafter. The development of live attenuated vaccines in general is difficult and time consuming. Moreover, fine-tuning the degree of attenuation is complex: high virulence causes disease, and low virulence induces insufficient protection. Surprisingly, vaccines according to the invention do not show significant differences in virulence when compared to their flagella-bearing counterparts. In other words, removal of the flagellin gene does not significantly change the level of attenuation. This has the unexpected advantage that both future and approved existing live attenuated Salmonella strains suitable for use in vaccines can be used in the present invention as soon as they are made non-capable to induce antibodies against at least one antigenic determinant of flagellin or flagella. Therefore, in another preferred form, the vaccines according to the invention comprise live attenuated bacteria.

Given the large amount of vaccines given to both pets and farm animals, it is clear that combined administration of several vaccines would be desirable, if only for reasons of decreased vaccination costs. It is therefore very attractive to use live attenuated bacteria as a recombinant carrier for heterologous genes, encoding antigens selected from other pathogenic microorganisms or viruses. Administration of such a recombinant carrier has the advantage that immunity is induced against two or more diseases at the same time. Live attenuated bacteria for use in a vaccine according to the present invention provide very suitable carriers for heterologous genes. In principle such heterologous genes can be inserted in the bacterial genome at any non-essential site.

Therefore, another embodiment of the invention relates to bacteria according to the invention in which a heterologous gene is inserted. Such a heterologous gene can, as mentioned above, for example, be a gene encoding an antigen selected from other pathogenic microorganisms or viruses. Such genes can, for example, be derived from pathogenic herpesviruses (for example, the genes encoding the structural proteins of herpesviruses), retroviruses (for example, the gp160 envelope protein), adenoviruses and the like.

Also a heterologous gene can be obtained from pathogenic bacteria. As an example, genes encoding bacterial toxins such as Actinobacillus pleuropneumoniae toxins, Clostridium toxins, outer membrane proteins and the like are very suitable bacterial heterologous genes.

Another possibility is to insert a gene encoding a protein involved in triggering the immune system, such as an interleukin, Tumor Necrosis Factor or an interferon, or another gene involved in immune-regulation.

The use of the flagellin gene as an insertion site has the advantage that there is no need to find a new insertion site for the heterologous gene and at the same time the flagellin gene is inactivated and the newly introduced heterologous gene can be expressed (in concert with the homologous bacterial genes). The construction of such recombinant carriers can be done routinely, using standard molecular biology techniques such as allelic exchange.

Thus, in a preferred embodiment, the heterologous gene is inserted in the flagellin gene. The heterologous gene can be inserted somewhere in the flagellin gene or it can be inserted at the site of the flagellin gene while this gene has been partially or completely deleted.

A vaccine according to the present invention also contains, in addition to the bacterium described above or antigenic material thereof, a pharmaceutically acceptable carrier. Such a carrier may be as simple as water, but it may, for example, also comprise culture fluid in which the bacteria were cultured. Another suitable carrier is, for example, a solution of physiological salt concentration.

The useful dosage to be administered will vary depending on the age, weight and animal vaccinated, the mode and route of administration and the type of pathogen against which vaccination is sought. The vaccine may comprise any dose of bacteria, sufficient to evoke an immune response. Doses ranging between 10³ and 10¹⁰ bacteria are, for example, very suitable doses.

Optionally, one or more compounds having adjuvant activity may be added to the vaccine. Adjuvants are non-specific stimulators of the immune system. They enhance the immune response of the host to the vaccine. Examples of adjuvants known in the art are Freunds Complete and Incomplete adjuvant, vitamin E, non-ionic block polymers, muramyldipeptides, ISCOMs (immune stimulating complexes, cf. for instance European Patent EP 109942), Saponins, mineral oil, vegetable oil, and Carbopol. Adjuvants, specially suitable for mucosal application are, for example, the E. coli heat-labile toxin (LT) or Cholera toxin (CT). Other suitable adjuvants are, for example, aluminum hydroxide, aluminum phosphate or aluminum oxide, oil-emulsions (for example, of Bayol F® or Marcol 52®), saponins or vitamin-E solubilisate.

Therefore, in a preferred form, the vaccines according to the present invention comprise an adjuvant.

Other examples of pharmaceutically acceptable carriers or diluents useful in the present invention include stabilizers such as SPGA, carbohydrates (for example, sorbitol, mannitol, starch, sucrose, glucose, dextran), proteins such as albumin or casein, protein containing agents such as bovine serum or skimmed milk and buffers (for example, phosphate buffer). Especially when such stabilizers are added to the vaccine, the vaccine is very suitable for freeze-drying or spray-drying.

Therefore, in a preferred form, the vaccine is in a freeze-dried or spray-dried form.

For administration to animals or humans, the vaccine according to the present invention can be given inter alia intranasally, by spraying, intradermally, subcutaneously, orally, by aerosol or intramuscularly. For application to poultry, wing web and eye-drop administration are additionally suitable.

Another embodiment of the invention relates to the use of bacteria according to the invention for the manufacture of a vaccine for the protection of humans and animals against infection with a Salmonella bacterium or the pathogenic effects of infection.

The invention also relates to methods for the preparation of a vaccine according to the invention. Such methods comprise the admixing of bacteria according to the invention or antigenic material thereof and a pharmaceutically acceptable carrier.

### DESCRIPTION OF THE DRAWINGS

FIG. 1: protein profile analysis of STM2000 and its parental strain S. typhimurium STMP. Lanes 1, 11 and 12 molecular weight markers as indicated; lanes 2, 5 and 8, S. typhimurium STMP 12× passed on blood agar medium; lanes 3, 6, and 9, STM2000 12× passed on blood agar medium; lanes 4, 7, and 10, STM2000 2× passed on blood agar medium. Lanes 2, 3, and 4, total protein profile; lanes 5, 6, and 7, pellet after sonication and centrifugation; lanes 8, 9 and 10, supernatant after sonication and centrifugation. Proteins were stained with Coomassie Brilliant Blue.

FIG. 2 Part (A): photograph of electron microscopical examination of S. typhimurium STMP; Part (B): photograph of electron microscopical examination of its non-motile mutant STM2000.

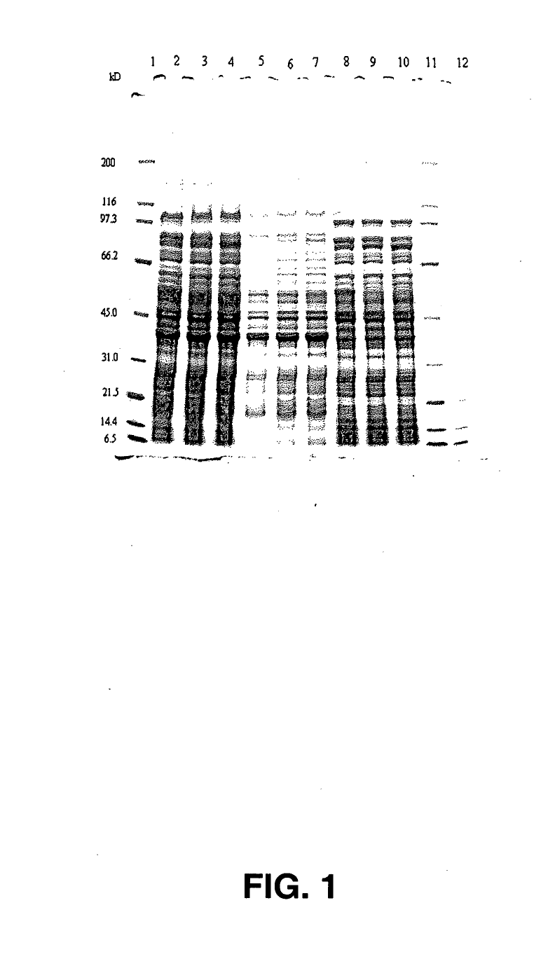

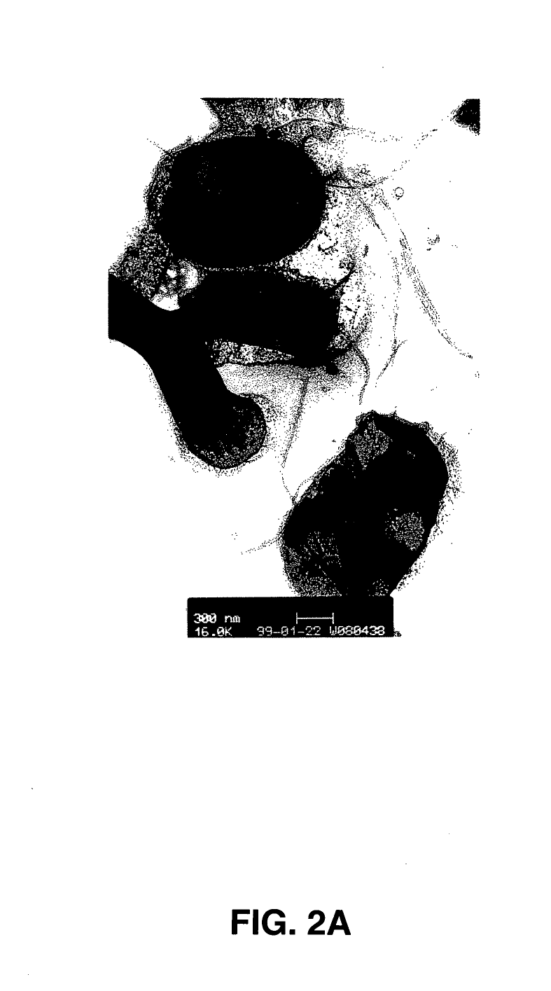

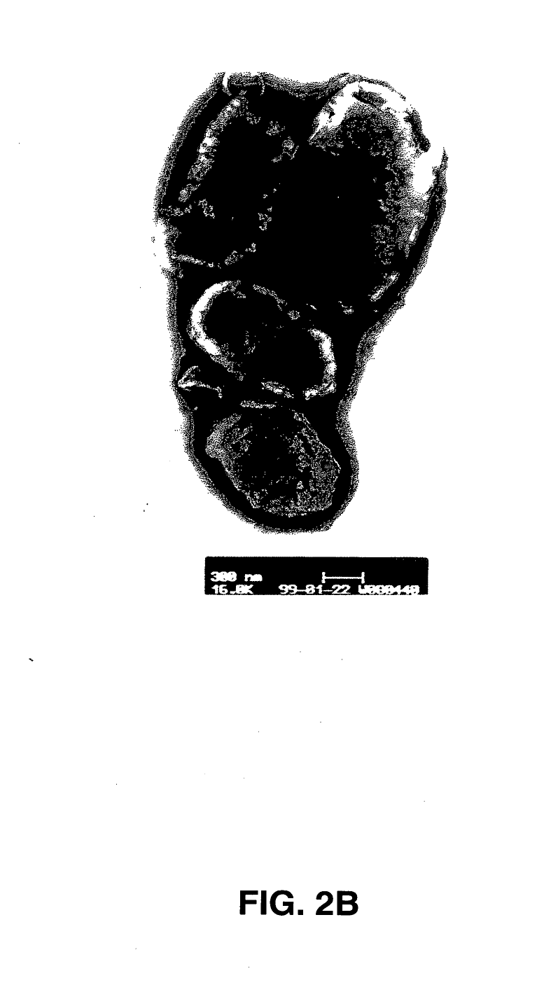

### DETAILED DESCRIPTION OF THE INVENTION

A diagnostic test for the screening for the absence/presence of Salmonella anti-flagellin or anti-flagella antibodies in sera can be a simple ELISA-test in which flagella or purified flagellin or a short polypeptide comprising an antigenic fragment thereof is coated to the wall of the wells of an ELISA-plate. Incubation with serum from humans or animals to be tested, followed by incubation with a labeled antibody against the relevant human or animal antibody can then reveal the presence or absence of antibodies against the flagellin.

Another example of a diagnostic test system is, for example, the incubation of a Western blot comprising flagellin with serum of humans or animals to be tested, followed by analysis of the blot.

The diagnostic tests for the detection of antibodies against Salmonella flagellin are preferably in the form of a kit, comprising flagellin in a purified form. The flagellin could be purified through standard protein separation techniques over a suitable column. Another possibility is separation by SDS-PAGE gel followed by Western-blotting. On the Western-blot, the flagellin will form a specific band, separated from other Salmonella protein bands, and thus is also considered to be purified. Also, a pure form of the protein can be obtained by expressing flagellin-encoding nucleic acid sequences.

In principle, the easiest way of making such a diagnostic test system is to use purified whole flagellin as explained above. It is however possible to use only part of the flagellin, with the proviso that the fragment used still comprises an antigenic determinant of the protein. All antigenic determinants of the flagellin will induce antibodies by definition. Therefore, the use of a flagellin fragment comprising even one single antigenic determinant of the flagellin will be capable of binding to anti-flagellin antibodies.

A test that is capable of discriminating between serum from non-infected, infected and vaccinated humans or animals comprises a well A, coated with flagellin and a well B, coated with another Salmonella immunogen, or possibly whole Salmonella cells. Serum that reacts with wells A and B indicates a field infection or vaccination with a classical vaccine, whereas serum that reacts only with well B indicates that the animal tested is vaccinated with the marker vaccine.

The invention may be further understood by reference to the non-limiting examples set forth below.

### EXAMPLES

### Example 1

Salmonella typhimurium strain STMP, an attenuated strain that has been tested as a live vaccine in poultry and pigs and provides good levels of protection was used as starting material for chemical mutagenesis.

Chemical Mutagenesis

S. typhimurium STMP was grown on blood agar medium and checked for a positive 0-antigen group B agglutination and H-antigen type 2 agglutination. One colony was inoculated into LB medium and incubated for 20 hours at 37° C. with aeration. Ten μl overnight culture was diluted in 10 ml LB (three cultures) and incubated at 37° C. for six hours with aeration until the culture reached an O.D. at 600 nm of 0.5. A sample was taken to determine the number of viable bacteria. To each of the three 10 ml cultures 100 μl trioxalen (chemical mutagens form Sigma; 3 mg/ml in DMSO) was added and the suspension was poured into a 10 cm Ø petri dish. The suspension was irradiated with U.V. (Transilluminator UVP; wavelength 365 nm) for 5, 10, or 15 minutes at a distance of 20 cm. Ten μl was transferred to 10 ml 0.9% NaCl, 1/10 dilutions were made and 100 μl was plated on blood agar plates in order to determine the survival rate of bacteria in mutagenized cultures. Cultures with survival rates of approximately 3% and 20% were grown in 100 ml LB until an OD at 600 nm of 0.5. Bacteria were collected by centrifugation, resuspended in 5 ml LB and stored in 30% glycerol at 70° C.

Selection for a Non-Motile, Aflagellated Mutant.

The mutagenized bacteria (3% survival; between 5,000 and 10,000 independent mutants) were inoculated from the glycerol stock onto three blood agar plates each. Bacteria were collected in LB and the OD at 600 nm was adjusted to 2.17. Six ten-fold dilutions were made in LB and then 50 μl H2-antiserum (Difco) was added to 450 μl bacterial suspension. This selection step with H-antiserum was included in order to enrich the suspension of mutants for aflagellated bacteria. This suspension was incubated at 37° C. for six hours while shaking (250 rpm) and then centrifuged for one minute at 1000 rpm in an Eppendorf mini centrifuge in order to remove the agglutination-complex. From the supernatant, 1, 10, and 100 μl was plated on blood agar plates and incubated 20 hours at 37° C.

Results:

Identification of a Non-Motile Mutant of S. typhimurium STMP.

Selection for non-flagellated bacteria by incubation with H2 antiserum resulted in growth of approximately 40 colonies on plates (10⁶ dilution) inoculated with the serum-treated mutant suspension (3% survival), whereas no colonies had grown on plates (10⁶ dilution) inoculated with the serum-treated STMP suspension. This result indicated that mutants were present that did not agglutinate with the H2-antiserum, possibly being aflagellated. The 40 colonies were tested for H2-agglutination and for motility using light microscopy. All mutants were negative for H2-agglutination, but only one mutant appeared non-motile as observed by light microscopy. This non-motile mutant was positive for group B O-antigen agglutination. The mutant was named STM2000.

In Vitro Stability of STM2000, a Non-Motile Mutant of S. typhimurium STMP.

The phenotypic stability of STM2000 was tested by twelve in vitro passages on blood agar plates. In the same experiment, the parent strain, STMP, was also passed twelve times. These cultures were observed by light microscopy and all bacteria in the mutant-culture were still non-motile. STMP bacteria were motile, although not all cells showed the same level of motility. Electron microscopic comparison of STMP and STM2000 confirmed that no flagella were present on mutant bacteria.

In Vitro Stability of STM2001, a Non-Motile Mutant of S. typhimurium STMP.

In a different experiment S. typhimurium SL3261 (Deposit number SGSC 439, Salmonella Genetic Stock Centre, University of Calgary, Alberta, Canada) was chemically mutagenized with NTG and non-motile mutants were selected as described before. One mutant, STM2001, showed no reaction with a flagellin-specific monoclonal antibody. After 2D protein gel electrophoresis it was shown that STM2001 lacked the flagellin spot of 51 kDa and pI 4.7, as compared to its parent strain.

The genetic stability of this strain was tested by growing for at least 50 generations. After this period, still no revertants were found: all bacteria were still non-motile.

S. typhimurium strain STM2001 has been deposited with the Centraalbureau voor Schimmelcultures (CBS), Uppsalalaan 8, 3584 CT, P.O. Box 85167, 3508 AD Utrecht, The Netherlands, under accession-number CBS 108955.

### Example 2

### Experimental Design

Vaccines were prepared from a flagellated and a non-flagellated S. enteritidis (“S.e.”) phage type 4 strain. The bacteria were cultured in Tryptose Phosphate Broth, inactivated by the addition of formalin to a final concentration of 0.5%, followed by harvest of the bacterial cells by centrifugation. The cells were resuspended in phosphate buffer saline and formulated into water in oil emulsion vaccines at 5×10⁹ bacteria/ml. Five chickens were injected intramuscularly with the S.e. fla⁺ vaccine and five chickens received the S.e. flat vaccine. The animals were vaccinated with 0.5 ml vaccine at 14 and 18 weeks of age. At 22 weeks of age, the chickens were bled, and serum was tested in a double antibody sandwich blocking ELISA system specific for antibodies to the g.m flagellin of S.e. (F. G. van Zijderveld et al. (1993) Vet. Quart. 15:135-137).

Animals

Commercial laying type chickens, approximately 14 weeks of age were obtained from a Salmonella free flock.

Results:

All five chickens that had received the S.e. fla⁺ vaccine seroconverted in the g.m specific ELISA (blocking percentage>60%) whereas the five chickens vaccinated with the fla⁻ vaccine remained seronegative.

### Example 3

### Experiment 1

### Experimental Design

To assess safety, broilers were inoculated orally (1 ml), subcutaneously (0.5 ml) and intramuscularly (0.5 ml) with flagella-positive Salmonella typhimurium strain STMP, flagella-negative Salmonella typhimurium strain STM2000 or wild-type S. typhimurium (Salmonella typhimurium). The animals were observed for one week after inoculation followed by post-mortem examination of the surviving chickens.

Animals

Commercial broilers, three weeks of age were obtained from a Salmonella free flock.

Results:

Following inoculation, eight out of ten animals that had received wild-type Salmonella typhimurium died (Table 1). At necropsy, the two surviving chickens inoculated with the wild-type strain had swollen livers with necrotic foci, swollen spleens and pericardial edema. One of the STMP inoculated chickens had a slightly swollen liver and one chicken inoculated with STM2000 had a slightly swollen spleen. No further abnormalities were noted in those two groups.

TABLE 1

| | Group | | |
|---|---|---|---|
| | STMP | STM2000 | Wild-type |
| Dose (CFU/ml) | 10^{8.0} | 10^{7.7} | 10^{8.0} |
| Mortality | 0/10^{a} | 0/10^{a} | 8/10^{b} |

Groups with different superscripts in a row differ (p < 0.5, Fisher exact test)

### Experiment 2

### Experimental Design

To test both safety and efficacy, broilers were inoculated orally at three and 15 days of age with either STMP or S™2000 followed by challenge infection with a tetracycline-resistant wild-type Salmonella typhimurium strain at 22 days of age.

Safety was assessed by clinical observation after vaccination and determination of weight gain. Also, cloacal swabs were taken at days 10 and 22 to determine the presence of the vaccine strains in the intestinal tract. Swabs were used to inoculate Brilliant Green Agars (BOA) directly and after enrichment in Rappaport Vassiliades Broth (RVB). The animals were observed for one week after challenge infection followed by postmortem examination of the surviving chickens. The livers, spleens, cloacal swabs and swabs of the caecum contents of the surviving vaccinated chickens were cultured for the challenge strain by direct inoculation on BGA containing tetracycline (BGAtet). In addition, the swabs were incubated in an enrichment medium (buffered peptone water containing tetracycline) followed by plating on BGAtet.

Animals

Commercial broilers, three days of age were obtained from a Salmonella free flock.

Results:

No clinical abnormalities were observed after the oral vaccinations at three and 15 days of age. Also, the average weight gains in the vaccinated groups were not different from the control group (Table 3). Both strains were present in cloacal swabs of the vaccinated animals taken seven days after vaccination (Table 2). The fact that a larger proportion of the chickens in the STMP inoculated group was culture positive after direct plating indicates that this strain colonizes the intestinal tract in higher numbers than the STM2000 strain.

TABLE 2

| | STMP positive swabs | | STM2000 positive swabs | |
|---|---|---|---|---|
| Reisolation | Direct | Enrichment | Direct | Enrichment |
| Day 10 | 15/15^{a} | Not done | 7/15^{b} | 14/15 |
| Day 22 | 10/15^{a} | 15/15^{A} | 14/15^{a} | 14/15^{A} |

Groups with different superscripts in a row differ (p < 0.5, Fisher exact test)

One chicken in the STMP group died after challenge infection (7%), compared to no mortality in the STM2000 group and 80% mortality in the unvaccinated controls (Table 3). The surviving chickens in the control group also gained considerable less weight than the vaccinates. The weight gain of the STM2000 vaccinated group after challenge infection was significantly higher than the weight gain of the STMP vaccinated group.

TABLE 3

| | Group | | |
|---|---|---|---|
| | STMP | SM2000 | Control |
| Dose d3 | 10^{8.8} | 10^{8.5} | — |
| Dose d15 | 10^{8.5} | 10^{8.5} | — |
| Weight gain d3-d22 | 636 ± 98^{a} | 594 ± 72^{a} | 624 ± 82^{a} |
| S. t. challenge dose d22 | 10^{8.0} | 10^{8.0} | 10^{8.0} |
| Weight gain d22-d29 | 299 ± 45^{a} | 339 ± 42^{b} | 31 ± 39^{c} |
| Mortality | 1/15^{a} | 0/15^{a} | 8/10^{b} |

Groups with different superscripts in a row differ (p < 0.5, Fisher exact test (mortality) or two-sample t test (weight gain).

As shown in Table 4, there was no difference in the reisolation rates of the challenge organism from the spleen and liver. Colonization of the intestinal tract, as judged by reisolation from cloacal swabs and caecum contents, was significantly lower in the group vaccinated with STM2000.

TABLE 4

| | Salmonella typhimurium (tet¹) positive | | | |
|---|---|---|---|---|
| | Group | | | |
| | STMP vaccinated | | STM2000 vaccinated | |
| Reisolation | Direct | Enrichment | Direct | Enrichment |
| Spleen | 2/14^{a} | Not done | 1/14^{a} | Not done |
| Liver | 1/14^{a} | Not done | 0/15^{a} | Not done |
| Cloaca | 7/14^{a} | 14/14^{A} | 3/15^{a} | 7/15^{B} |
| Caecum | 13/14^{a} | 14/14^{A} | 5/15^{b} | 9/15^{B} |

Groups with different superscripts in a row differ (p < 0.5, Fisher exact test).

### Example 4

### Experimental Design

To test efficacy, pigs were inoculated orally with STMP (10^{9.0} CFU) or S™2000 (10^{9.3} CFU) followed by an oral challenge infection with a streptomycin-resistant wild-type Salmonella typhimurium strain (10^{11.0} CFU) two weeks later.

Shedding of the challenge strain was measured by culturing fecal samples on days 5 and 8 post challenge. Approximately 5 grams of feces were placed in peptone water and incubated for two hours. Then, serial ten-fold dilutions were made in RVB medium containing streptomycin, incubated overnight, followed by plating on Tryptose Phosphate Broth containing streptomycin. The maximum dilution containing the challenge strain was used to calculate the reisolation score (reciprocal of the maximum dilution positive). Eight STMP vaccinated pigs and eight unvaccinated controls were used in a first experiment. In a second experiment, nine pigs were vaccinated with STM2000 and nine unvaccinated controls were used.

Animals

Six-week-old SPF pigs were used in both experiments.

Results:

As shown in Table 5, both vaccine strains were able to reduce fecal shedding of the challenge strain significantly.

TABLE 5

Average reisolation score

| | Day 5 | Day 8 |
|---|---|---|
| STMP (n = 8) | 10^{0.9a} | 10^{1.0a} |
| Control (n = 8) | 10^{6.4b} | 10^{3.8b} |
| STM2000 (n = 9) | 10^{1.0A} | 10^{1.3A} |
| Control (n = 9) | 10^{5.0B} | 10^{4.9B} |

Groups with different superscripts differ (p < 0.5, Mann-Whitney U test).

1. A method for reducing the colonization of wild-type flagella carrying Salmonella enterica in a subject, the method comprising:
administering to the subject a composition comprising Salmonella enterica bacteria and a pharmaceutically acceptable carrier so as to reduce the colonization of wild-type flagella carrying Salmonella enterica in the subject as compared to a subject that has not been administered the composition, the Salmonella enterica bacteria comprising a mutated bacterium that in its wild type form carried flagella having at least one antigenic determinant, but in its mutated form is no longer capable of inducing an immune response to the at least one antigenic determinant of flagellin in a subject to which the composition is administered.

2. The method according to claim 1, wherein the Salmonella enterica bacteria lacks at least one antigenic determinant of flagellin or flagella due to a mutation in a gene of the flagellar biogenesis pathway.

3. The method according to claim 1, wherein administering to the subject a composition comprises administering to the subject a composition comprising Salmonella enterica bacteria, a pharmaceutically acceptable carrier and an adjuvant.

4. The method according to claim 1, wherein administering to the subject a composition comprises administering to the subject a composition comprising Salmonella enterica in freeze-dried or spray-dried form.

5. The method according to claim 1, wherein administering to the subject a composition comprises administering to the subject a composition comprising Salmonella enterica in live attenuated form.

6. The method according to claim 1, wherein administering to the subject a composition comprises administering to the subject a composition comprising Salmonella enterica in inactivated form.

7. The method according to claim 1, wherein administering to the subject a composition comprises administering the composition to a chicken or a pig.

8. A method of marking an exposure of a subject to wild-type Salmonella, the method comprising:
administering an immunogenic composition comprising an immunologically effective amount of live mutated bacteria and a pharmaceutically acceptable carrier to a subject;
wherein, said live mutated bacteria are Salmonella enterica that in their wild-type form carried flagella having at least one antigenic determinant; and
wherein after mutation, said live mutated bacteria are not capable of inducing an immune response to the at least one antigenic determinant of flagellin so as to allow marking of an exposure of the subject to the wild-type Salmonella in the subject to which it is administered.

9. The method according to claim 8, further comprising administering an adjuvant to the subject.

10. The method according to claim 9, wherein administering the adjuvant further comprises selecting the adjuvant selected from the group consisting of Freunds Complete adjuvant, Freunds Incomplete adjuvant, vitamin E, non-ionic block polymers, muramyldipeptides, immune stimulating complexes, saponins, mineral oil, vegetable oil, Carbopol, E. coli heat-labile toxin, Cholera toxin, aluminum hydroxide, aluminum phosphate, aluminum oxide, oil-emulsions, and vitamin-E solubilisate.

11. The method according to claim 8, wherein the pharmaceutically acceptable carrier is selected from the group consisting of water, a solution of physiological salt concentration, SPGA, sorbitol, mannitol, starch, sucrose, dextran, albumin, casein, bovine serum, skim milk, and phosphate buffer.

12. A method of marking of an exposure of a subject to wild-type Salmonella, the method comprising:
administering an immunologically effective amount of mutated bacteria, the mutated bacteria having a mutation in a gene encoding flagellin, and a pharmaceutically acceptable carrier to a subject;
wherein said mutated bacteria are Salmonella that in their wild-type form carried flagella having at least one antigenic determinant; and
wherein said mutated bacteria are not capable of inducing an immune response to the at least one antigenic determinant of flagellin in a subject to which it is administered.

13. The method according to claim 12, wherein the mutated bacteria comprise Salmonella typhimurium, wherein the wild type form of the mutated S. typhimurium carries flagella;

14. The method of claim 12, wherein the mutated bacteria comprise Salmonella typhimurium strain STMP.

15-25. (canceled)
